(19) United States
(12) Patent Application Publication
(10) Pub. No.: US 2010/0234905 A1
(43) Pub. Date: Sep. 16, 2010

(54) FACET JOINT RESURFACING IMPLANT AND ASSOCIATED SURGICAL METHODS

(76) Inventors: John SLEDGE, Marblehead, MA (US); Trace Cawley, Boca Raton, FL (US)

Correspondence Address: Clements Bernard PLLC, 1901 Roxborough Road, Suite 250, Charlotte, NC, 28211, US

(21) Appl. No.: 12/723,173

(22) Filed: Mar. 12, 2010

Related U.S. Application Data
(60) Provisional application No. 61/160,179, filed on Mar. 13, 2009.

(51) Int. Cl. A61B 17/56

(52) U.S. Cl. 606/86 A

(57) ABSTRACT

The present invention provides devices and methods for resurfacing a facet joint of a spine of a patient, including: a first portion that is selectively coupled to an opposed surface of a superior facet of the facet joint of the spine of the patient; and a second portion that is selectively coupled to an opposed surface of an inferior facet of the facet joint of the spine of the patient; wherein the first portion is allowed a predetermined degree of movement with respect to the second portion. These devices and methods find applicability to other anatomical joints as well.

### CROSS-REFERENCE TO RELATED APPLICATION

The present non-provisional patent application/patent claims the benefit of priority of U.S. Provisional Patent Application No. 61/160,179, filed on Mar. 13, 2009, and entitled “FACET RESURFACING DEVICE AND ASSOCIATED METHODS,” the contents of which are incorporated in full by reference herein.

### FIELD OF THE INVENTION

The present invention relates generally to devices and methods for performing surgical procedures, such as spinal surgical procedures. More specifically, the present invention relates to devices and methods for selectively flexing a facet joint of a spine or other similar joint or bony structure, resurfacing the associated facets or other similar bony structures, and, optionally, holding the facet joint or other similar joint in a flexed, compressed, and/or distracted configuration.

### BACKGROUND OF THE INVENTION

Facet joints of the spine are in almost constant motion and often wear out or become degenerated or damaged. When facet joints become worn or torn, the associated cartilage may become thin or disappear, and there may be reactionary contact of the underlying bone, producing an overgrowth of bony spurs and enlargement of the facet joints. Such a facet joint presents arthritic changes, or osteoarthritis, that may result in significant back pain with motion. This condition is often referred to as “facet joint disease” or “facet joint syndrome.” Similar diseases and syndromes are experienced in relation to other joints of the human body.

Facet joint disorders are among the most common of all of the recurrent, disabling back and neck problems, and may cause a significant degree of discomfort and disability for patients. Degeneration of the adjoining intervertebral disc is almost always present, such the affected segment often requires a fusion procedure of some sort. In such a fusion procedure, adjacent vertebrae are immobilized while an implanted bone graft is allowed to “take,” for example, using a conventional pedicle screw system, a plate system, or the like and, optionally, an intervertebral cage of some sort. Pedicle screw systems consist of a plurality of pedicle screws that are anchored to adjacent levels of the spine and connected with stabilizing rods or the like. Plate systems consist of a plate that is anchored to adjacent levels of the spine and, optionally, connected to the intervertebral cage or other implantable device.

In the treatment of facet joint disorders, and other similar joint disorders, it is often desirable to resurface the facets of the facet joint, counteracting the development of bony spurs and enlargement of the facet joint. In order to accomplish this, the superior and inferior facets of the facet joint must be selectively flexed for drilling/preparation and implant placement, for example. Conventional devices and methods would make this a tricky process, which is never desirable during a surgical procedure.

Thus, what are needed in the art are simple and efficient devices and methods for selectively flexing the facet joint of the spine or other similar joint or bony structure, resurfacing the associated facets or other similar bony structures, and, optionally, holding the facet joint or other similar joint in a flexed, compressed, and/or distracted configuration. Optionally, the facet joint resurfacing implant would allow for some movement of the facet joint or other similar joint, replicating the natural movement of the human back or neck, or other anatomical structure.

### BRIEF SUMMARY OF THE INVENTION

In various exemplary embodiments, the present invention provides simple and efficient devices and methods for selectively flexing the facet joint of the spine or other similar joint or bony structure, resurfacing the associated facets or other similar bony structures, and, optionally, holding the facet joint or other similar joint in a flexed, compressed, and/or distracted configuration. Optionally, the facet joint resurfacing implant allows for some movement of the facet joint or other similar joint, replicating the natural movement of the human back or neck, or other anatomical structure.

In one exemplary embodiment, the present invention provides a device for resurfacing a facet joint of a spine of a patient, including: a first portion that is selectively coupled to an opposed surface of a superior facet of the facet joint of the spine of the patient; and a second portion that is selectively coupled to an opposed surface of an inferior facet of the facet joint of the spine of the patient; wherein the first portion is allowed a predetermined degree of movement with respect to the second portion. Optionally, the first portion is allowed a predetermined degree of translational movement with respect to the second portion. Optionally, the first portion is allowed a predetermined degree of compressive movement with respect to the second portion. Optionally, the first portion is allowed a predetermined degree of expansive movement with respect to the second portion. Optionally, the first portion is allowed no movement with respect to the second portion. Optionally, the first portion and the second portion are integrally formed. Optionally, the first portion and the second portion are each formed of a substantially pliable material. Optionally, the first portion and the second portion are each formed of a substantially rigid material. Optionally, the first portion and the second portion each include a facet-engaging portion and an intra-facet portion. Optionally, the facet-engaging portion of each of the first portion and the second portion include one or more of a protruding structure, a recessed structure, a friction structure, and a bonding surface that is configured to engage one or more of a corresponding recessed structure, protruding structure, friction structure, and bonding surface formed into/on one of the superior facet and the inferior facet, respectively.

In another exemplary embodiment, the present invention provides a device for resurfacing a bony joint of a patient, including: a first portion that is selectively coupled to an opposed surface of a first bony structure of the bony joint of the patient; and a second portion that is selectively coupled to an opposed surface of a second bony structure of the bony joint of the patient; wherein the first portion is allowed a predetermined degree of movement with respect to the second portion.

In a further exemplary embodiment, the present invention provides a method for resurfacing a facet joint of a spine of a patient, including: selectively coupling a first portion to an opposed surface of a superior facet of the facet joint of the spine of the patient; and selectively coupling a second portion to an opposed surface of an inferior facet of the facet joint of the spine of the patient; wherein the first portion is allowed a predetermined degree of movement with respect to the second portion. Optionally, the first portion is allowed a predetermined degree of translational movement with respect to the second portion. Optionally, the first portion is allowed a predetermined degree of compressive movement with respect to the second portion. Optionally, the first portion is allowed a predetermined degree of expansive movement with respect to the second portion. Optionally, the first portion is allowed no movement with respect to the second portion. Optionally, the first portion and the second portion are integrally formed. Optionally, the first portion and the second portion are each formed of a substantially pliable material. Optionally, the first portion and the second portion are each formed of a substantially rigid material. Optionally, the first portion and the second portion each include a facet-engaging portion and an intra-facet portion. Optionally, the facet-engaging portion of each of the first portion and the second portion include one or more of a protruding structure, a recessed structure, a friction structure, and a bonding surface that is configured to engage one or more of a corresponding recessed structure, protruding structure, friction structure, and bonding surface formed into/on one of the superior facet and the inferior facet, respectively.

### BRIEF DESCRIPTION OF THE DRAWINGS

The present invention is illustrated and described herein with reference to the various drawings, in which like reference numbers are used to denote like device components/method steps, as appropriate, and in which:

FIG. 1 is a top view illustrating the facet joint of the spine;

FIG. 2 is a top view illustrating the facet joint of the spine in a flexed configuration;

FIG. 3 is a top view illustrating the facet joint of the spine after preparation/modification, with a recess being formed in the opposed faces of each of the superior facet and the inferior facet in accordance with the methods of the present invention;

FIG. 4 is a top view illustrating the facet joint of the spine with one exemplary embodiment of the facet joint resurfacing implant of the present invention installed therein in accordance with the methods of the present invention;

FIG. 5 is a side view illustrating the facet joint of the spine with one exemplary embodiment of the facet joint resurfacing implant of the present invention installed therein in accordance with the methods of the present invention; and

FIG. 6 is a flowchart illustrating one exemplary embodiment of the method for surgically implanting the facet joint resurfacing implant of the present invention.

### DETAILED DESCRIPTION OF THE INVENTION

In various exemplary embodiments, the present invention provides simple and efficient devices and methods for selectively flexing the facet joint of the spine or other similar joint or bony structure, resurfacing the associated facets or other similar bony structures, and, optionally, holding the facet joint or other similar joint in a flexed, compressed, and/or distracted configuration. Optionally, the facet joint resurfacing implant allows for some movement of the facet joint or other similar joint, replicating the natural movement of the human back or neck, or other anatomical structure.

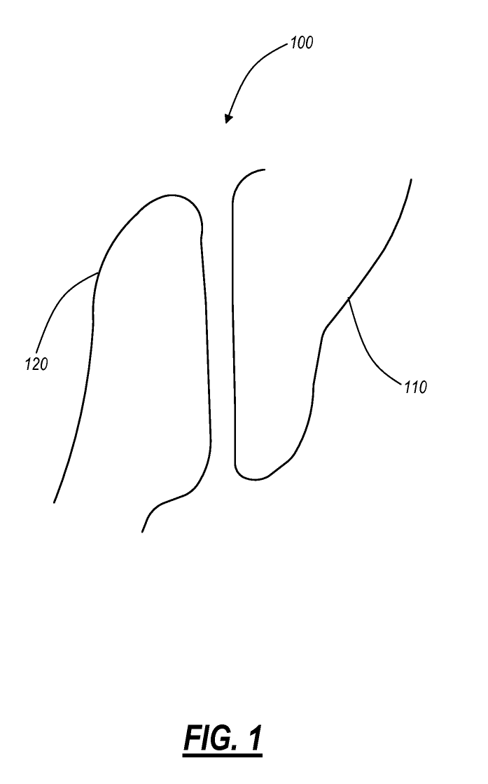

Referring to FIG. 1, the facet joint 100 of the spine includes a superior facet 110 and an inferior facet 120. In an otherwise healthy facet joint 100, cartilage (not illustrated) is typically disposed between the superior facet 110 and the inferior facet 120. When the facet joint 100 becomes worn or torn, this cartilage may become thin or disappear, and there may be reactionary contact of the underlying bone, i.e. the superior facet 110 and the inferior facet 120, producing an overgrowth of bony spurs and enlargement of the facet joint 100. It will be readily apparent to those of ordinary skill in the art, that although a facet joint is illustrated and described herein, the devices and methods of the present invention are applicable to any similar joint or other anatomical structure.

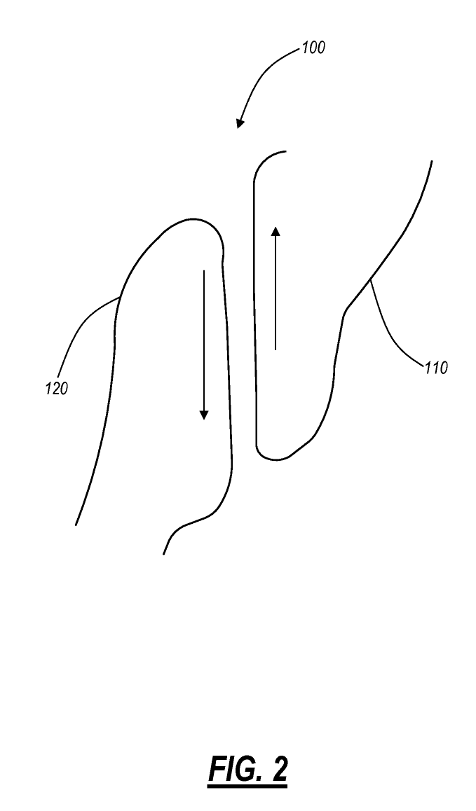

Referring to FIG. 2, the facet joint 100 is illustrated after it is flexed or otherwise distracted (in any desired direction along any desired plane) in accordance with the methods of the present invention. In the example provided, the superior facet 110 is translated with respect to the inferior facet 120 along a given plane between them, as illustrated in the top view. Preferably, the facets 110, 120 are held in a spaced-apart relationship prior to the resurfacing step. This translation and spacing may be achieved using any techniques known or developed in the art.

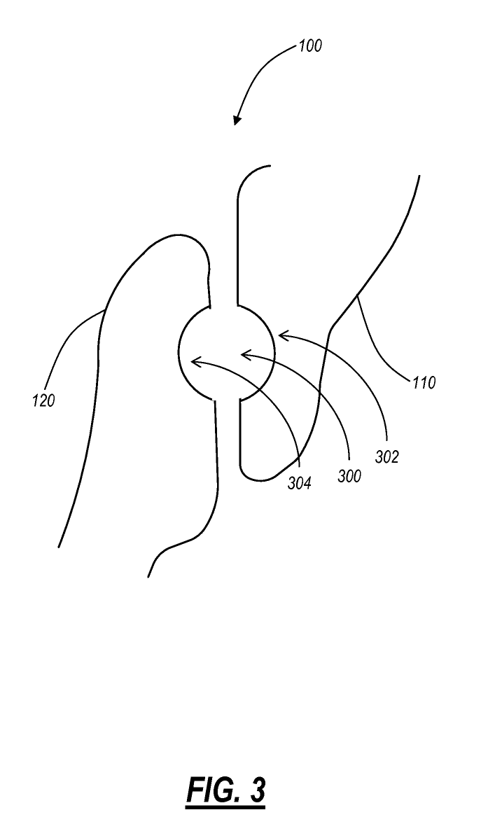

Referring to FIG. 3, in one exemplary embodiment, a hole 300 is drilled across the facet joint 100, creating a corresponding recess 302, 304 in the opposed faces of each of the facets 110, 120. Again, this hole 300 may be drilled in any desired direction along any desired plane, and may be formed to any desired depth or degree. This recess formation may be achieved using any techniques known or developed in the art. The inter-facet space may be opened up and a hole may be drilled across it, multiple holes may be drilled across it, multiple other recesses may be formed by grasping, cutting, and biting instruments such as rongeurs, rotating burrs, osteotomes, chisels, etc., or friction surfaces may simply be created—provided the opposed surfaces of the superior facet 110 and the inferior facet 120 are adequately prepared to receive the facet joint resurfacing implant of the present invention.

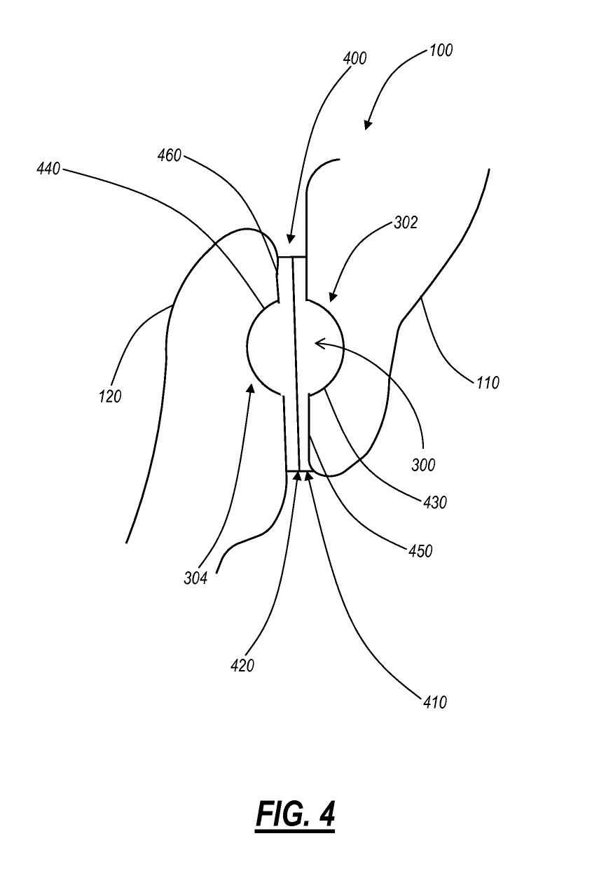

Referring to FIG. 4, in one exemplary embodiment, the facet joint resurfacing implant 400 of the present invention may include one or more pieces. For example, the facet joint resurfacing implant 400 may include a first portion 410 that is selectively coupled to the superior facet 110 of the facet joint 100 and a second portion 420 that is selectively coupled to the inferior facet 120 of the facet joint 100. The first portion 410 may glide across the second portion 420 in operation, may frictionally engage the second portion 420 in operation, may be joined to the second portion 420 mechanically or via an intermediate material (e.g. via a material having a different rigidity and/or modulus of elasticity), may be bonded to the second portion 420, may be integrally formed with the second portion, etc. In various exemplary embodiments, the goal is to provide a desired degree of movement in one or more given directions between the opposed faces of the superior facet 110 and the inferior facet 120. In other exemplary embodiments, the goal is to minimize such movement. The facet joint resurfacing implant 400 and its constituent components may be made of any surgically implantable material(s), such as a metal, a ceramic, a polymer, a biological material, etc., and should be appropriately dimensioned for implantation in a joint or other anatomical structure of interest.

The facet joint resurfacing implant 400 is selectively disposed in the opened up, flexed, distracted, compressed, translated, or otherwise positioned facet joint 100 and, to a desired degree, holds the facet joint 100 in that configuration once placed, while providing a desired degree of movement or “give,” from no movement to extensive movement, all depending on the configuration and material(s) of the facet joint resurfacing implant 400. Typically, the first portion 410 of the facet joint resurfacing implant 400 and the second portion 420 of the facet joint resurfacing implant 400 each include a facet-engaging portion 430, 440 that includes one or more protrusions or the like corresponding to the one or more recesses manufactured into the opposed faces of the facets 110, 120, a friction surface corresponding to the friction surfaces manufactured into the opposed faces of the facets 110, 120, or are otherwise anchored or bonded to the opposed faces of the facets 110, 120. It should be noted that, in all of the above descriptions, mating recesses and protrusions may be interchanged. In any case, the first portion 410 of the facet joint resurfacing implant 400 and the second portion 420 of the facet joint resurfacing implant 400 are securely seated on the opposed faces of the corresponding facets 110, 120, such that the bulk of the facet joint resurfacing implant 400 is held between the opposed faces and they are held in desired alignment(s), even with movement.

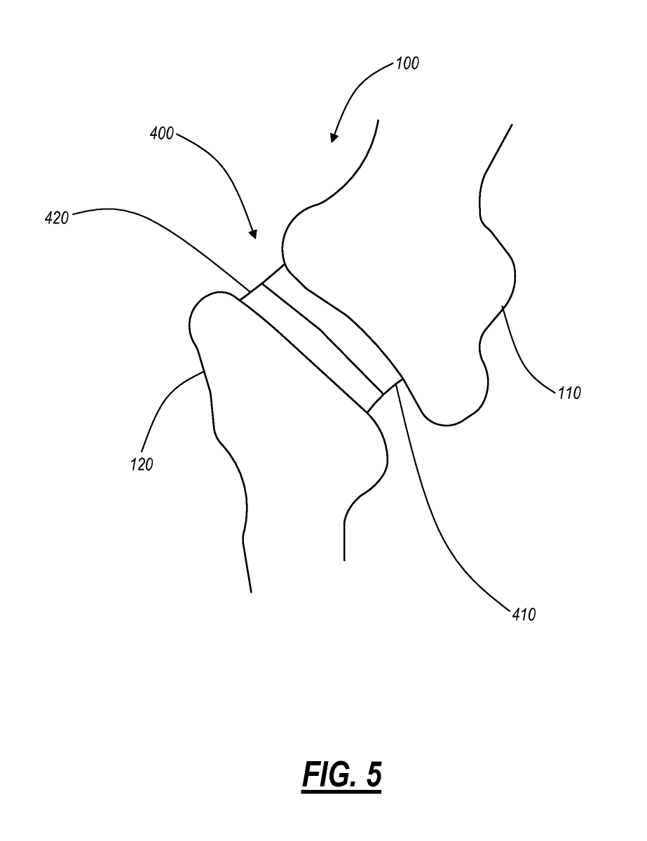

Referring to FIG. 5, a side view of the modified, resurfaced facet joint 100 is illustrated according to an exemplary embodiment of the present invention. Optionally, the first portion 410 is allowed a predetermined degree of sliding or translational movement with respect to the second portion 420. Again, optionally, the first portion 410 and the second portion 420 are integrally formed. The first portion 410 and the second portion 420 may be configured to absorb compressive forces between the superior facet 110 and the inferior facet 120.

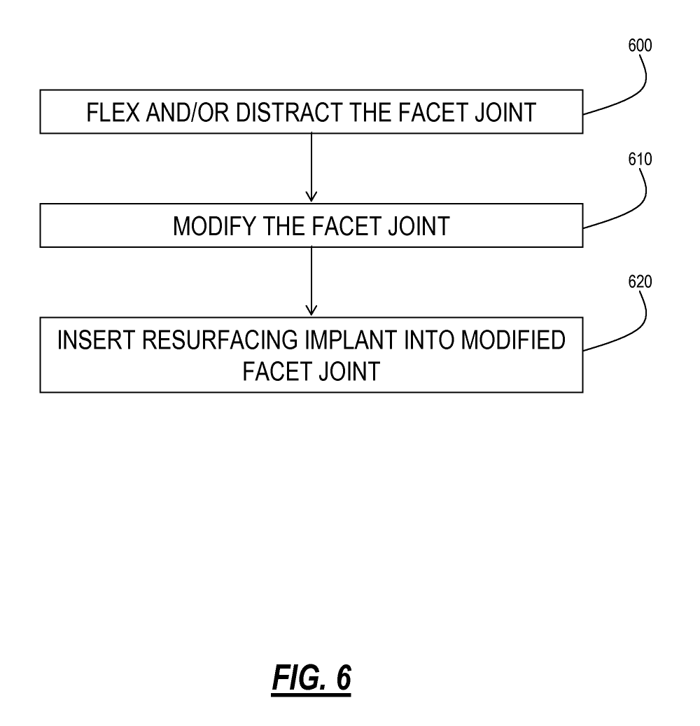

Referring to FIG. 6, a flowchart of the overall method for resurfacing a facet joint is illustrated according to an exemplary embodiment of the present invention. The first step involves flexing, distracting, and/or translating the facet joint so that the facet joint is maintains a desired configuration 600. The second step involves modifying the facet joint so that it is able to receive a “plug” or device, such as the facet joint resurfacing implant of the present invention 610. According to an exemplary embodiment of the present invention, the modification step may include drilling a joint-spanning hole or the like. The third step involves inserting the “plug” or device into the modified facet joint 620. The “plug” or device may allow for constrained movement of the facet joint, or may hold the facet joint essentially motionless.

Although the present invention has been illustrated and described herein with reference to preferred embodiments and specific examples thereof, it will be readily apparent to those of ordinary skill in the art that other embodiments and examples may perform similar functions and/or achieve like results. For example, although spinal surgical applications have been illustrated and described herein, the devices and methods of the present invention find equal applicability to a wide range of surgical applications, involving a wide range of anatomical joints. All such equivalent embodiments and examples are within the spirit and scope of the present invention, are contemplated thereby, and are intended to be covered by the following claims.

What is claimed is:

1. A device for resurfacing a facet joint of a spine of a patient, comprising:
a first portion that is selectively coupled to an opposed surface of a superior facet of the facet joint of the spine of the patient; and
a second portion that is selectively coupled to an opposed surface of an inferior facet of the facet joint of the spine of the patient;
wherein the first portion is allowed a predetermined degree of movement with respect to the second portion.

2. The device of claim 1, wherein the first portion is allowed a predetermined degree of translational movement with respect to the second portion.

3. The device of claim 1, wherein the first portion is allowed a predetermined degree of compressive movement with respect to the second portion.

4. The device of claim 1, wherein the first portion is allowed a predetermined degree of expansive movement with respect to the second portion.

5. The device of claim 1, wherein the first portion is allowed no movement with respect to the second portion.

6. The device of claim 1, wherein the first portion and the second portion are integrally formed.

7. The device of claim 1, wherein the first portion and the second portion are each formed of a substantially pliable material.

8. The device of claim 1, wherein the first portion and the second portion are each formed of a substantially rigid material.

9. The device of claim 1, wherein the first portion and the second portion each comprise a facet-engaging portion and an intra-facet portion.

10. The device of claim 9, wherein the facet-engaging portion of each of the first portion and the second portion comprise one or more of a protruding structure, a recessed structure, a friction structure, and a bonding surface that is configured to engage one or more of a corresponding recessed structure, protruding structure, friction structure, and bonding surface formed into/on one of the superior facet and the inferior facet, respectively.

11. A device for resurfacing a bony joint of a patient, comprising:
a first portion that is selectively coupled to an opposed surface of a first bony structure of the bony joint of the patient; and
a second portion that is selectively coupled to an opposed surface of a second bony structure of the bony joint of the patient;
wherein the first portion is allowed a predetermined degree of movement with respect to the second portion.

12. A method for resurfacing a facet joint of a spine of a patient, comprising:
selectively coupling a first portion to an opposed surface of a superior facet of the facet joint of the spine of the patient; and
selectively coupling a second portion to an opposed surface of an inferior facet of the facet joint of the spine of the patient;
wherein the first portion is allowed a predetermined degree of movement with respect to the second portion.

13. The method of claim 12, wherein the first portion is allowed a predetermined degree of translational movement with respect to the second portion.

14. The method of claim 12, wherein the first portion is allowed a predetermined degree of compressive movement with respect to the second portion.

15. The method of claim 12, wherein the first portion is allowed a predetermined degree of expansive movement with respect to the second portion.

16. The method of claim 12, wherein the first portion is allowed no movement with respect to the second portion.

17. The method of claim 12, wherein the first portion and the second portion are integrally formed.

18. The method of claim 12, wherein the first portion and the second portion are each formed of a substantially pliable material.

19. The method of claim 12, wherein the first portion and the second portion are each formed of a substantially rigid material.

20. The method of claim 12, wherein the first portion and the second portion each comprise a facet-engaging portion and an intra-facet portion.

21. The method of claim 20, wherein the facet-engaging portion of each of the first portion and the second portion comprise one or more of a protruding structure, a recessed structure, a friction structure, and a bonding surface that is configured to engage one or more of a corresponding recessed structure, protruding structure, friction structure, and bonding surface formed into/on one of the superior facet and the inferior facet, respectively.
